(19) United States
(12) Patent Application Publication
(10) Pub. No.: US 2011/0300234 A1
(43) Pub. Date: Dec. 8, 2011

(54) Methods of Treating Tumor Cells Using RHCC Protein, Fragment or Variant

(76) Inventors: Jürgen Müller, Bad Krozingen (DE); Egbert Figgemeier, Leichlingen (DE)

(21) Appl. No.: 13/128,952

(22) PCT Filed: Nov. 11, 2009
(86) PCT No.: PCT/IB2009/055015

§ 371 Date: Aug. 23, 2011

Related U.S. Application Data
(60) Provisional application No. 61/113,658, filed on Nov. 12, 2008.

(51) Int. Cl. A61K 33/24; A61P 35/00; A61K 38/02; A61K 31/282; A61K 31/44

(52) U.S. Cl. 424/649; 514/492; 514/332; 514/19.3

(57) ABSTRACT

Methods of treating tumor cells in an individual comprise administering to the individual a drug having anti-tumor effect, wherein the drug is delivered into the tumor cells via Right-Handed Coiled-Coil (RHCC) protein or a fragment or variant thereof. The drug may, for example, be a metal-containing compound, a protein or peptide drug, and/or an organic hydrophobic compound.

### FIELD OF THE INVENTION

The present invention is related to methods of treating tumor cells in an individual and particularly to methods wherein a drug having anti-tumor effect is delivered into the tumor cells via Right-Handed Coiled-Coil (RHCC) protein or a fragment or variant thereof.

### BACKGROUND OF THE INVENTION

Many drugs have been developed and others are under development for treatment of tumor cells. These drugs, inter alia, provide cytotoxic effects to tumor cells via various mechanisms. However, it is not uncommon for in vivo administration of an anti-tumor drug to provide adverse side effects in an individual undergoing treatment, for example, owing to delivery of the anti-tumor drug to normal cells in addition to the tumor cells. Such adverse side effects limit, inter alia, individuals to whom such drugs may be administered, dosing regimens and/or treatment durations, resulting in additional complications and/or ineffective tumor treatment.

As an example, the platinum-containing chemotherapeutic drug Cis-diammine-dichloroplatinum (II) (cisplatin) is one of the most potent and curative anti-tumor drugs available. Cisplatin is very effective in the treatment of testicular carcinoma, and is also used for treatment of ovarian, cervical, head and neck, non-small-cell lung cancer, bladder, and stomach cancers. See, for example, Boulikas et al, “Recent clinical trials using cisplatin, carboplatin and their combination chemotherapy drugs (review),” Oncol. Rep., 11(3):559-95 (2004). In fact, more than about one half of all cancer patients treated using chemotherapy receive platinum complexes. Cisplatin exerts its anticancer effect by several mechanisms, including formation of DNA adducts and production of reactive oxygen species. However, cisplatin causes a number of adverse effects, including serious and dose-limiting effects such as nephrotoxicity, ototoxicity and neurotoxicity, of which the latter two are usually irreversible. More specifically, neurotoxicity causes distal sensory neuropathy, while the ototoxic side effects are observed as sensorineural hearing loss, beginning in the high frequencies and involving successively lower frequencies toward the speech frequency range, and also tinnitus. See, for example, van der Hulst et al, “High frequency audiometry in prospective clinical research of ototoxicity due to platinum derivatives,” Ann. Otol. Rhinol. Laryngol., 97(2 Pt 1):133-7 (1988) and Nagy et al, “Cisplatin ototoxicity: the importance of baseline audiometry,” Am J Clin Oncol, 22(3):305-8 (1999).

Accordingly, it would be extremely advantageous to provide a more precise manner of anti-tumor drug delivery to tumor cells while minimizing delivery to normal cells and/or reducing adverse side effects of such drugs in any individual undergoing treatment.

### SUMMARY OF THE INVENTION

It is therefore an object of the present invention to provide methods for treating tumor cells.

In one embodiment, the invention is directed to a method of treating tumor cells in an individual, which method comprises administering to the individual a drug having anti-tumor effect, wherein the drug is delivered into the tumor cells via Right-Handed Coiled-Coil (RHCC) protein or a fragment or variant thereof.

In another embodiment, the invention is directed to a method of treating tumor cells in an individual, which method comprises administering to the individual a platinum-containing drug having anti-tumor effect, wherein the drug is delivered into the tumor cells via RHCC protein or a fragment or variant thereof and in the absence of an added tag.

Surprisingly, the RHCC protein or fragment or variant thereof has been found to penetrate tumor cells and therefore can be used for administration into the cells. Thus, the RHCC protein or fragment or variant thereof can be used to deliver the anti-tumor drug into the tumor cells, while substantially maintaining the anti-tumor effect of the drug. This can result in increased efficacy for a constant dose of anti-tumor drug and/or a reduction in delivery of the anti-tumor drug to normal cells, along with a reduction in adverse effects in an individual caused by delivery of the anti-tumor drug to normal cells. These and additional aspects and advantages of the present methods will be more apparent in view of the following detailed description.

### BRIEF DESCRIPTION OF THE DRAWINGS

The following detailed description will be more fully understood in view of the drawings, in which:

FIG. 1 shows a structural model of the RHCC protein, with a side view of the tetrameric channel, where the backbone is shown in ribbon representation for each helical chain, and the four cavities.

FIGS. 2A-2D show kinetics and dose-response of AF-RHCC-binding to FaDu cells as described in the Example, with FIGS. 2A and 2B showing binding, as measured by percent fluorescent cells and mean fluorescent intensity per positive cell, respectively, after 10 min to 8 hrs at 4° C. or 37° C., and FIGS. 2C and 2D showing binding, as measured by percent fluorescent cells and mean fluorescent intensity per positive cell, respectively, after incubation with 0.1-100 μg AF-RHCC at 37° C.

FIGS. 3A-3E show AF-RHCC uptake and cellular localization in FaDu cells as described in the Example in the form of fluorescent microscope photographs after incubation for 10 min (FIG. 3A), 60 min (FIG. 3B), 4 hr (FIG. 3C), and 8 hr (FIG. 3D) at 4° C. or 37° C. and a confocal laser scanning microscope photograph (FIG. 3E) after incubation for 8 hr at 37° C. Darker gray represents DAPI-staining of DNA in the nucleus, while the lighter gray and white represent AF-RHCC.

FIG. 4 shows the in vitro cytotoxic effects in 10 tumor cell lines as described in the Example by concentration-effect curves of (▴) RHCC, (▪) cisplatin, and () RHCC/cisplatin (RHCC/C). Cell survival is presented as survival index (SI %) which is defined as the fluorescence in experimental wells in percent of that in control wells, with blank values subtracted. Results are presented as mean±SEM of 3 independent experiments.

FIG. 5 shows the in vitro cytotoxic effects in primary human tumor cells from 3 patients (OC1-OC3) diagnosed with ovarian cancer as described in the Example by concentration-effect curves of (▴) RHCC, (▪) cisplatin and () RHCC/C. Cell survival is again presented as survival index (SI %) as defined above. Results are presented as mean±SEM of 3 independent experiments.

FIG. 6 shows in vivo tumor reduction from RHCC/C and cisplatin as described in the Example. Mean tumor size in mice treated with (▪) cisplatin, () RHCC/C, or (▴) NaCl. Results are presented as mean±SEM.

FIGS. 7A-7D show immune response in mice after RHCC injection, and in dendritic cells (DCs) after co-culture. Specifically, FIG. 7A shows the number of IFNγ-secreting cells per 106 murine splenocytes, FIG. 7B shows RHCC-specific antibody titres in murine serum, FIG. 7C shows the mean fluorescence of 4 different antibodies specific for surface maturation markers CD40, CD80, CD86 and MHC class II in murine DCs, and FIG. 7D shows IL-12 levels in serial diluted cell supernatants from murine DCs. Results are presented as mean±SEM.

The drawings thus illustrate specific features of an exemplary embodiment of certain aspects of the invention.

### DETAILED DESCRIPTION

The methods of the invention are directed to treating tumor cells in an individual and comprise administering to the individual a drug having anti-tumor effect wherein the drug is delivered into the tumor cells via Right-Handed Coiled-Coil (RHCC) protein or a fragment or variant thereof.

RHCC protein is part of the Tetrabrachion complex which constitutes the surface layer of the cell envelope of the archaebacteria Staphylothermus marinas, a bacterium living in the environment of so-called “black smokers” on the sea ground. The bacterium is sulphur dependent, has an optimal growth temperature of 92° C., and lives on the fermentation of peptides. The bacterium has a quasi-periplasmic space containing so-called stacks, which are built of four-stranded helical coiled-coils together with two proteases. The bacterium, Tetrabrachion and the RHCC protein all exhibit extreme thermostability and strong resistance against denaturants. Specifically, the RHCC protein has been shown to be stable in high salt concentrations, at temperatures of over 100° C., at high pressures, and over extreme ranges of pH.

The structure of the RHCC protein has been established by x-ray crystallography and comprises an α-helical domain made up of four strands oriented in parallel in a right-handed fashion. Each of the four RHCC strands contains 52 amino acid residues of the sequence GSIINETADDIVYRLTVIIDDRYESLKNLITLRADRLEMIINDNVSTILASI (SEQ ID NO:1) and comprises the protease-binding region of tetrabrachion (amino acid residues 1238-1287). RHCC is nano-sized and has an average length of 72 Å and an average diameter of 25 Å and a molecular weight of 22.8 kDa. While the outside of the protein structure is rather hydrophilic, the inside has strong hydrophobic character and a large buried surface of roughly 9500 Å². The crystal structure of the protein shows an axial channel through the entire tetramer, which is accompanied by four large cavities. The cavities have volumes ranging from 145 to 300 Å³ and strong hydrophobic character. In the crystal structure, these cavities are filled with water molecules.

Within the present disclosure, RHCC protein refers to a peptide comprising four strands, sometimes called peptide strands, together forming the right-handed parallel RHCC tetramer, the sequence of at least one of said peptide strands being of SEQ ID NO:1. In the 52 amino acid sequence of SEQ ID NO:1, 11-residue repeat positions, indicated by lowercase letters, have been assigned according to the following model proposed by Lupas (Trends Biochem. Sci., 21:375-382 (1996)) and Stetefeld et al (Nature Struct. Biol., 7(9):772-776 (2000)), both of which are incorporated herein by reference:

| | a | b | c | d | e | f | g | h | i | j | k |
|---|---|---|---|---|---|---|---|---|---|---|---|
| 1-3 | | | | | | | | | G | S | I |
| 4-11 | I | N | E | T | A | D | D | I | | | |
| 12-18 | | | | | V | Y | R | L | T | V | I |
| 19-29 | I | D | D | R | Y | E | S | L | K | N | L |
| 30-40 | I | T | L | R | A | D | R | L | E | M | I |
| 41-51 | I | N | D | N | V | S | T | I | L | A | S |
| 52 | I | | | | | | | | | | |

Hydrophobic core positions are a and h. The continuity of the 11-residue repeats is interrupted by a four-residue insertion (stutter) between Ile 11 and Thr 16. The first two N-terminal residues, Gly 1 and Ser 2 are not part of the Tetrabrachion coding sequence. The native RHCC starts with Ile. The GS terminal residue originates from the vector used in recombinant production, for example, as described by Stetefeld et al (2000) and does not belong to the Tetrabrachion protein. As described experiments have been performed with the recombinantly expressed protein starting with GS, this sequence is indicated herein. In a fusion molecule, these amino acids can function as a linker. Numbering of the amino acids is indicated on the left of the model set forth above. Amino acid residue Ile 3 corresponds to position 1238 on the Tetrabrachion sequence. Accordingly, in the present context, the term “RHCC protein” refers to a tetrameric complex comprising four peptide strands, at least one of which is of SEQ ID NO:1 or aa 3-52 of SEQ ID NO:1. In specific embodiments of the present invention, two, three, or four of the peptide strands are of SEQ ID NO:1. It is furthermore to be understood that an “RHCC protein” may also refer to a crystal complex of several RHCC proteins, or a RHCC protein with one or more fragments thereof.

The term “RHCC protein fragment” or the term “fragment thereof' in the phrase “RHCC protein or fragment thereof' refers to a portion of an RHCC protein. More particularly, a “fragment” according to the invention, comprises any suitable amino acid fragment of any length of an RHCC protein which exhibits the desired functionality of the RHCC protein, namely, the ability to penetrate tumor cells. Accordingly, a fragment of an RHCC protein may in the context of the present invention comprise at least one part of one peptide strand of a tetrameric RHCC protein, up to at least one part of the four strands of an RHCC protein. An RHCC protein fragment in one embodiment according to the invention, comprises up to 207 amino acids, such as, but not limited to, about 50 to 100, 100 to 150, 150 to 200, or 200 to 207 amino acids. A fragment of an RHCC protein may also be longer if any of the RHCC peptide strands has been extended with additional amino acid residues, which are not part of the amino acid sequence of an RHCC peptide strand (SEQ ID NO:1). The amount of additional amino acid residues to be added to the natural sequence is not limited, it is however envisaged that a binding domain may encompass about 100-150 residues. A fragment will also include a crystal complex of at least two RHCC protein fragments as described above.

Encompassed by the present invention is also the use of an RHCC protein and/or a fragment thereof, which has been modified in any suitable manner, for example to form a variant of an RHCC and/or a fragment thereof, that is still able to deliver a desired anti-tumor drug as described hereafter. More specifically, a variant of an RHCC protein and/or fragment thereof, is an RHCC protein or fragment which has been point mutated, extended by adding any suitable amount of amino acids to the RHCC protein and/or fragment, such as, but not limited to, between 1-5 and 5-10 amino acids, while still exhibiting the ability to deliver the anti-tumor drug into tumor cells. It should be understood that any modifications as disclosed may be performed in the nucleic acid and/or amino acid sequence of one or more of the RHCC peptide strands and/or fragments thereof.

In one embodiment, the variant is an amino acid sequence being at least 70% identical, such as being at least 72%, 75%, 77%, 80%, 82%, 85%, 87%, 90%, 91%, 92%, 93%, 94%, 95%, 96%, 97%, 98%, or 99% identical with the amino acid sequence of a RHCC peptide and/or a fragment thereof according to the invention. A protein, polypeptide, peptide and/or a fragment thereof having an amino acid sequence at least, for example, 90% identical to a reference amino acid sequence, is intended that the amino acid sequence is identical to the reference sequence, except that the amino acid sequence may include up to 10 point mutations per each 100 amino acids, of a reference amino acid sequence. In other words, to obtain a peptide having an amino acid sequence e.g. at least 90% identical to a reference amino acid sequence, up to 10% of the amino acids in the reference sequence may be deleted or substituted with another amino acid, or a number of amino acids. These mutations of the reference sequence may occur at the amino or carboxy terminal positions of the reference amino acid sequence or anywhere between those terminal positions, interspersed either individually among amino acids in the reference sequence or in one or more contiguous groups within the reference sequence. One or more point mutations in the sequence of one or more of the four individual strands of an RHCC protein and/or a fragment thereof may be made to improve the functionality of the RHCC protein such as, but not limited to, the delivery of the anti-tumor drug into tumor cells. Consequently, variant includes an RHCC protein and/or a fragment thereof, which has been point mutated at any suitable position in a RHCC protein and/or fragment thereof, to obtain an improved functionality. Any suitable amount of point mutations may be used to obtain an RHCC protein and/or a fragment thereof with desired functionality. In one aspect of the invention, a point mutation is performed in at least one peptide strand of an RHCC protein and/or a fragment thereof.

A variant of the RHCC protein and/or a fragment thereof may further include the addition of a tag, i.e. the addition of an amino acid sequence, e.g., a peptide or fragment thereof, including an antibody or fragment thereof, a carbohydrate and/or a chemical substance, to one or more of the RHCC proteins and/or fragments thereof and/or to a RHCC peptide strand, achieved through joining the coding sequence of RHCC and any other peptide or protein within an expression vector when producing a RHCC protein and/or fragment thereof in a living organism, or achieved through the binding by chemical means to one or more of the peptide strands of a RHCC protein during or after the synthesis. Such a tag may be associated to a single RHCC protein and/or to a fragment thereof and/or to a complex of RHCC proteins and/or fragments thereof of at least two RHCC proteins. However, as will be discussed in further detail below, the use of a tag is, surprisingly, not necessary to deliver an anti-tumor drug into tumor cells in accordance with the present methods.

In the present invention, a local algorithm program is best suited to determine identity. Local algorithm programs, (such as Smith-Waterman) compare a subsequence in one sequence with a subsequence in a second sequence, and find the combination of subsequences and the alignment of those subsequences, which yields the highest overall similarity score. Internal gaps, if allowed, are penalized. Local algorithms work well for comparing two multidomain proteins, which have a single domain or just a binding site in common. Methods to determine identity and similarity are codified in publicly available programs Preferred computer program methods to determine identity and similarity between two sequences include, but are not limited to, the GCG program package (Devereux et al (1994)) BLASTP, BLASTN, and FASTA (Altschul et al (1990)). The BLASTX program is publicly available from NCBI and other sources (BLAST Manual, Altschul et al (1990)). Each sequence analysis program has a default scoring matrix and default gap penalties. In general, a molecular biologist would be expected to use the default settings established by the software program used.

An RHCC protein and/or a fragment or variant thereof can be produced in a number of different ways, including, but not limited to, fragmentation of larger molecules, chemical synthesis, recombinant technology, and/or by a combination of these methods. It is to be understood by the person skilled in the art, that any method for producing an RHCC protein and/or a fragment or variant thereof may be used in the context of the present invention. In one embodiment, the RHCC protein, fragment or variant is produced recombinantly. In a more specific embodiment, the RHCC protein is produced recombinantly according to the technique described in the Example. In a specific embodiment, the RHCC protein, fragment or variant is substantially isolated. Such a substantially isolated or purified form will generally comprise the protein or fragment in a preparation and/or a composition in which more than approximately 90%, e.g. 95%, 96%, 97%, 98%, 99% or 100% of the proteins, polypeptides, and/or peptides in the preparation is the RHCC protein fragment or variant. Additionally, the RHCC protein, fragment or variant may be mixed with one or more carriers or diluents which do not interfere with the desired anti-tumor treatment.

In the remainder of the description, reference to the RHCC protein should be understood to include reference to a fragment or variant thereof, as well, unless otherwise indicated.

In accordance with the present methods, a drug having an anti-tumor effect is administered to the individual. An anti-tumor effect may comprise, for example, a cytotoxic effect to the tumor cells, retardation of tumor growth, and/or a reduction in tumor size, or other tumor treatment result. The drug having anti-tumor effect may be selected from numerous such drugs known in the art. Examples include, but are not limited to, metal-containing drugs, peptide and protein drugs, small molecule drugs, for example organic hydrophobic compounds, alkylating agents, antimetabolites, and the like.

In one embodiment, the drug comprises a metal-containing drug, i.e., an anti-tumor drug having a metal element therein. The term “metal element” refers to an element which comprises a metal constituent of any kind and/or amount, such as atoms of metals and/or half metals, as well as any applicable substance containing such, whether in charged or non-charged, hydrophobic, hydrophilic or any other form. In a specific embodiment of the invention, an element is not only selected from metal elements, but from the group consisting of: Al, Sc, Ti, V, Cr, Fe, Co, Ni, Cu, Zn, Ga, Ge, As, Se, Sr, Y, Zr, Nb, Mo, Tc, Ru, Rh, Pd, Ag, Cd, In, Sn, Sb, Te, Cs, Ba, La, Ce, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, At, Fr, Ra, Ac, Th, Pa, U, Np, Pu, Am, Cm, Bk, Cf, Es, Fm, Md, No, Lr, Rf, Db, Sg, Bh, Hs and Mt, and/or a combination thereof.

In a more specific embodiment, the metal-containing drug comprises a platinum-containing drug. As discussed, platinum drugs have been relatively successful in exhibiting anti-tumor effects. In a yet more specific embodiments of the invention, the metal-containing drug comprises at least one platinum-containing drug selected from the group consisting of cisplatin, carboplatin, nedaplatin, oxaliplatin, ZD0473, JM216, lobaplatin, trans-[PtCl₂(pyridine)₂] and trans-[Pt(OH)₂Cl₂ (NH₃)(NH₂—C₂H₅)]. In a further embodiment, the drug comprises cisplatin.

In further embodiments, the anti-tumor drug comprises one or more organic compounds, examples of which include, but are not limited to, actinomycins, e.g., dactinomycin, anthracyclines, e.g., doxorubicin and idarubicin, taxanes, e.g., paclitaxel and docetaxel, antibiotics, e.g., calicheamicin, tyrosine kinase inhibitors, e.g., sunitinib, sorafenib, lapatinib, dasatinib, erlotinib, and gefitinib, cyclin dependent kinase inhibitors, e.g., seliciclib, and histone deacetylase inhibitors, e.g., vorinostat, and sphingosine.

In accordance with a further embodiment of the invention, a combination of anti-tumor drugs may be administered. Additionally, in yet a further embodiment, an anti-tumor drug may be administered in combination with a second active agent providing a different therapeutic effect to the target cells. Suitable combinations of anti-tumor drugs and/or combinations of one or more anti-tumor drugs with one or more other active agents for use in these embodiments will be apparent to one of ordinary skill in the art.

The anti-tumor drug is associated with the RHCC protein or fragment or variant thereof prior to administration of the drug to the individual. The association may be by incorporation of the anti-tumor drug in the cavities of the RHCC protein or fragment or variant structure or may be by bonding of the anti-tumor drug to the RHCC protein or fragment or variant thereof. Incorporation of the drug in the cavities of the RHCC protein or a fragment thereof is particularly appropriate for small drugs, for example metal-containing compounds such as the platinum anti-tumor drugs discussed above, along with small non-metal compounds, for example, sphingosine. Without wishing to be bound by theory, energy-driven processes are believed to assist in locating and maintaining metal-containing compounds within the cavities. Contacting a solution of drug, for example, an NaCl solution of the drug, with the RHCC protein or fragment or variant for a sufficient time can result in the desired association.

Various bonding techniques may also be employed. For example, the anti-tumor drug may be covalently bound to the outside of the RHCC protein or fragment, provided such binding does not inactivate the anti-tumor properties of the drug. In one embodiment, for example, a small molecule, protein or peptide anti-tumor drug is bonded to either the C- or the N-terminal of the RHCC protein or fragment. In one embodiment, the drug is bound to the N- or C-terminal by covalent bonding. Without being bound by theory, it is believed the RHCC protein may first enter the cells with the N- or C-terminal end, whereby delivery of the anti-tumor drug bound at the entering end may facilitate delivery of the drug within the cell. In another embodiment, the drug may bind, for example, to outer amine groups on the RHCC protein. For example, each RHCC protein strand contains an external Lysine, whereby four drug molecules could bind to the four external Lysine groups. Linking groups, for example, a His linking group, may also be employed to facilitate bonding of the drug. The ratio of drug molecule to RHCC protein or fragment may therefore vary. In one embodiment, the ratio is about 1:1. In another embodiment, the ratio of drug molecule to RHCC protein or fragment is in a range of from about 1:1 to about 20:1, more specifically from about 1:1 to about 10:1, or more specifically from about 1:1 to about 5:1. In further embodiments, the ratio is about 1:1, 2:1, 3:1 or 4:1.

As is demonstrated in the Example herein, the RHCC protein enters the cells to a great extent, thereby facilitating delivery of the anti-tumor drug. This is surprising in that in many conventional technologies, a tagging molecule has typically been used to deliver an anti-tumor drug to the vicinity of tumor cells and/or facilitate entry of the drug into the cells, often with only limited success. Additionally, according to the present methods, the anti-tumor drug's anti-tumor effects are substantially maintained, i.e., an anti-tumor effect is exhibited in the tumor cells. The ability of the RHCC protein to deliver the anti-tumor drug into the tumor cells, in the absence of a tag, while maintaining the anti-tumor effect of the drug, provides significant advantages, including, for example, increased efficacy for a particular dose of anti-tumor drug as more drug will typically be delivered into the tumor cells, and/or reduced delivery of anti-tumor drug to normal cells, and/or a reduction in adverse side effects from a particular dose of drug, and/or the like. Additionally advantages of the RHCC protein delivery of an anti-tumor drug into tumor cells will be apparent to those of ordinary skill in the art. Without being bound by theory, it is believed that these advantages may be due, in part, to the “enhanced permeability and retention (EPR) effect” wherein tumor vessels are “leaky” and allow macromolecular extravasation and tumors lack effective lymphatic drainage, preventing clearance of macromolecules and promoting their accumulation in the tumor. Further, again without being bound by theory, it is believed that the increased size of the anti-tumor drug in association with the RHCC protein or fragment thereof, as compared with the anti-tumor drug itself, may reduce the likelihood of certain adverse side effects which are caused by penetration of blood-organ barriers. For example, the increased size of cisplatin in association with RHCC protein may reduce cisplatin-induced ototoxicity, since the larger combination is more unlikely to penetrate the blood-perilymph barrier separating the inner ear from the systemic circulation, a system similar to the blood-brain barrier.

The present methods may be used for treatment of various types of tumor cells. In one embodiment, the methods are suitable for treatment of sarcoma, carcinoma, lymphoma or myeloma tumor cells. In another specific embodiment, the tumor cells comprise ovarian carcinoma, testicular cancer, head tumor, neck tumor, breast cancer, colorectal cancer, urinary bladder cancer, renal cancer, small cell lung cancer, or non-small cell lung cancer cells. As demonstrated in the Example, the present methods provide improved cytotoxic effects in various tumor cell types.

The anti-tumor drug, in association with the RHCC protein or fragment or variant thereof, may be administered to the individual via any known route. In a specific embodiment, the drug is administered intraveneously. In a more specific embodiment, a platinum-containing drug in association with the RHCC protein or a fragment or variant thereof is administered intravenously. In another embodiment, the anti-tumor drug, in association with the RHCC protein or a fragment, or variant thereof may be administered to the individual intraperitoneally. In a more specific embodiment, a platinum-containing drug in association with the RHCC protein or a fragment or variant thereof is administered intraperitoneally. Depending on the anti-tumor drug which is employed, in specific embodiments, the anti-tumor drug, in association with the RHCC protein or a fragment or variant thereof, may be administered via other conventional administration routes, including oral, parenteral, buccal, aural, rectal, vaginal, topical or nasal. The anti-tumor drug is used in its recommended dosage, although the dosage may, in some embodiments, be reduced to obtain the same or better anti-tumor effect, as delivery of the drug into the tumor cells may improve efficacy.

The following Example demonstrates various aspects of specific embodiments of the invention.

### EXAMPLE

The following procedures are employed:

Production and Purification of RHCC

Recombinant RHCC polypeptide chain fragments are produced in E. coli according to the procedures of Stetefeld et al (2000). A synthetic gene encoding residues Ile 3-Ile 52 of the Tetrabrachion sequence is ligated into the BamH/EcoRI site of pet15b, and expressed in E. coli BL21(DE3) (Novagen, Darmstadt, Germany). RHCC is purified from bacterial lysates by Ni—NTA Sepharose affinity chromatography (Qiagen, Hilden, Germany) under denaturing conditions, and refolded in physiological buffer conditions on the Ni—NTA column. The polyhistidine tag is cleaved using thrombin and the cleaved peptide removed by Ni—NTA. RHCC solution is purified from bacterial endotoxins by incubation with Polymyxin B-agarose (Sigma-Aldrich, Stockholm, Sweden) at 4° C. for 1 hr with rotation. Protein concentration is measured using NanoDrop (NanoDrop Technologies, Wilmington, Del., USA). Purified RHCC ranges from 10-15 mg/L culture.

Incorporation of Cisplatin into RHCC

RHCC and cisplatin (Mayne, Warwickshire, UK) are mixed using 1 mg of each at room temperature for 1 hr, and then centrifuged at 14 krpm for 5 min to remove undissolved cisplatin. The supernatant is run on a PD-10 desalting column according to the manufacturer's instructions (Amersham Biosciences, Uppsala, Sweden) to remove unbound cisplatin. Protein concentration is measured by NanoDrop, and platinum concentrations are measured by inductively-coupled plasma optical emission spectrometry (ICP-OES) at 214.424 nm RHCC with incorporated cisplatin is denominated RHCC/C.

Conjugation of RHCC with Alexa Fluor 488 5-Sulfodichlorophenol Ester

RHCC is conjugated to Alexa Fluor 488 5-sulfodichlorophenol ester (SDP) according to the manufacturer (Invitrogen, Carlsbad, Calif., USA). RHCC is mixed with Alexa Fluor 488 SDP reactive dye for 1 hr, and conjugated protein is separated from free dye on a PD-10 desalting column. The Alexa Fluor 488 SDP:protein ratio is ˜1.5 moles dye per mole protein as measured by NanoDrop. RHCC conjugated to Alexa Fluor 488 SDP is denominated AF-RHCC.

Flow Cytometry

5×104 FaDu-cells, a head and neck squamous cell carcinoma line (see Rangan, “A new human cell line (FaDu) from a hypopharyn-geal carcinoma,” Cancer, 29(1): 117-21 (1972)) in 300 μl cell culture medium are incubated with 0.1-100 μg of AF-RHCC for 10 min-8 hours, at 4° C. or 37° C., washed twice in PBS and analyzed by flow cytometry in a FACS Calibur using Cell Quest software (Becton Dickinson, San Jose, Calif., USA).

Fluorescent and Confocal Laser Scanning Microscopy

5×104 FaDu-cells/well seeded in 300 μl FaDu-medium overnight at 37° C. in 8-chamber microscopy slides (BD Biosciences, San Diego, Calif., USA) are mixed with 100 μg AF-RHCC for 10 min-8 hours, at 4° C. or 37° C. Cells are washed 3 times in PBS, fixed in 3% paraformaldehyde (Sigma-Aldrich, Stockholm, Sweden) for 15 min, washed 3 times, and mounted with Vectashield HardSet medium with DAPI (Immunkemi, Jarfalla, Sweden). Cells are photographed at ×40 magnification in a Zeiss Axioplan 2 microscope with Zeiss AxioVision software (Carl Zeiss AB, Stockholm, Sweden), and ×60 magnification in a Nikon Eclipse TE 300 confocal laser scanning microscope (Carl Zeiss AB, Stockholm, Sweden) with Ultra View software (Perkin Elmer, Waltham, Mass., USA).

Human Cell Lines and Primary Human Tumor Cells (PHTC)

FaDu is grown in DMEM and 10% heat-inactivated FBS, 0.1 mM MEM non-essential amino acids, 1.2 mM sodium pyruvate, 24 mM HEPES, with penicillin and streptomycin. MDA 231, a breast cancer cell line, RPMI 8226, a myeloma cell line, its resistant sub-line 8226/Dox40, NCI-H69, a small lung cancer cell line, its resistant sub-line H69AR, ACHN, a primary resistant renal adenocarcinoma, the ovarian carcinoma cell line A2780, and its resistant sub-line, A2780-Cis, are all grown in RPMI-1640 with 10% heat-inactivated FCS, 2 mM glutamine, penicillin and streptomycin. hTERT-RPE1 a normal epithelial telomerase immortalized line is grown in DMEM nutrient mixture F-12 Ham with 10% heat-inactivated FCS, 2 mM glutamine, penicillin and streptomycin. Tumor cells obtained from 3 ovarian carcinoma patients (OC1-OC3) after ethical approval (Uppsala University ethical committee) are isolated by collagenase dispersion and Percoll density gradient centrifugation (GE Healthcare Life Sciences, Uppsala, Sweden) (see Csoka et al, Gynecol. Oncol., 54(2):163-70 (1994)). Cells (minimum 70% viability) are frozen in FCS with 10% DMSO (Sigma-Aldrich, Stockholm, Sweden) for 24 hr in −70° C. and stored at −150° C., which does not affect drug sensitivity (see Nygren et al, Leukemia, 6(11):1121-8 (1992)). For experiments, frozen cells are thawed, washed twice and kept in RPMI 1640.

Fluorometric Microculture Cytotoxicity Assay (FMCA)

RHCC, RHCC/C, and cisplatin are tested in triplicates at 6 concentrations by 2-fold serial dilution in 0.1 M NaCl, starting at 3.48 μM for RHCC and RHCC/C (with 10 μM cisplatin), and 10 μM for cisplatin. Microtitre plates (Nunc, Roskilde, Denmark) are prepared with 20 μl/well of drug solution 10 times the desired concentration. To evaluate the cytotoxic activity, the drug plates are seeded with 2×104 cells/180 μl/well. A column without drugs serves as a control, and a column with medium serves as a blank FMCA is based on measurements of fluorescence generated from hydrolysis of fluorescein diacetate (FDA) to fluorescein by cells with intact plasma membrane (see Larsson et al, Int. J. Cancer, 50(2):177-85 (1992)). The plates are incubated at 37° C. for 72 hr, washed in PBS, whereafter FDA (dissolved in PBS, 10 μg/ml) is added at 100 μl/well. Plates are then incubated for 50 min, and fluorescence/well is measured at 538 nm in a scanning fluorometer (Fluoroscan II, Labsystems Oy, Helsinki, Finland). Fluorescence is proportional to the number of viable cells/well. Quality criteria for successful analysis includes a fluorescence signal in control wells of more than 5 times mean blank value and a mean coefficient of variation in control wells of less than 30%. Experiments were repeated 3 times.

Animals

Balb/c mice in open cages and SCID mice in individually ventilated cages according to the MAC III IVC-system were bred and kept at MTC, Karolinska Institute.

In Vivo Tumor Reduction Assay

SCID-mice are injected s.c. with 5×105 FaDu-cells in 100 μl PBS, and one week later injected i.v. with 1.0 mg/kg cisplatin, 0.675 mg RHCC/C (with ˜0.35 mg/kg cisplatin); or 0.1 M NaCl (5 mice/group). In a separate experiment 5 mice are after tumor challenge treated with 0.675 mg RHCC. Mice are palpated and weighed 3 times/week, and euthanized if the tumor diameter exceeded 10 mm or if weight decreased below 80% of starting weight, according to ethical guidelines.

Enzyme-Linked Immunosorbent Spot (EliSpot) Assay

IFN-γ EliSpot assays are performed according to the manufacturer's instructions (Mabtech, Nacka, Sweden). Splenocytes (1.2×105) from Balb/c-mice collected 7 days after i.v. injection of 0.4 or 0.2 mg RHCC (2 mice/group), or 0.1 M NaCl (one control mouse) are cultured in triplicates for 40 hr in anti-mouse IFN-γ antibody coated EliSpot plates (Millipore AB, Solna, Sweden) alone, or with 1, 5 or 10 μg/ml of RHCC, an LCMV derived peptide NP118-126 (RPQASGVYM) (SEQ ID NO:2) (see Shen et al, Cell, 92(4):535-45 (1998)) as negative control, or phorbol myristate acetate (25 ng/ml) and ionomycin (250 ng/ml) (Sigma-Aldrich, Stockholm, Sweden) as positive control. Spots are counted in an EliSpot reader, and processed by EliSpot Reader 4.0 software (Autoimmun Diagnostika GmbH, Strassberg, Germany).

Enzyme-Linked Immunosorbent Assay (ELISA)

The antibody response to RHCC in sera from Balb/c-mice collected 14 days after i.v. injection of 0.4 or 0.2 mg RHCC (2 mice/group), or 0.1 M NaCl (one mouse), is measured by ELISA. Microtitre plates (BD Biosciences, San Diego, Calif., USA) are coated with 5 μg/ml RHCC in 0.1 M carbonate-buffer pH 9.6 per well o.n. at 4° C. Plates are blocked in blocking solution (5% milk powder, 0.2% Tween in PBS) for 1 hr at room temperature. Serial dilutions of mouse sera in blocking solution (1:50-1:1350) is added in duplicates to the wells. After 1 hr at room temperature, plates are washed and incubated with secondary alkaline phosphatase (AP) conjugated goat-anti mouse IgG antibody (Sigma-Aldrich, Stockholm, Sweden). Plates are washed and developed with nitrophenylphosphate (pNPP) (Sigma-Aldrich, Stockholm, Sweden) and absorbance is measured at 405 nm in a VersaMax microplate reader (Molecular Devices, Sunnyvale, Calif., USA).

Dendritic Cell (DC) maturation Assay

Murine bone marrow-derived dendritic cells (BMDCs) are generated according to Lutz et al, J. Immunol. Methods, 223(1):77-92 (1999), and murine splenic DCs are purified using CD11c (N418) MicroBeads (Miltenyi Biotec, Auburn, Calif., USA) according to the manufacturer. DCs are seeded in 24-well plates (BD Biosciences, San Diego, Calif., USA) at 106 cells/ml in R10 medium and mixed with 50 μg/ml RHCC. LPS 1 μg/ml (Sigma-Aldrich, Stockholm, Sweden) is used as positive control. After 24 hr stimulation, DCs are stained with PE-conjugated antibodies to CD40, CD80 and CD86 and FITC-conjugated antibody to MHC Class II I-Ab (BD Biosciences Pharmingen, San Diego, Calif., USA) 30 min at 4° C., washed twice in PBS with 0.1% BSA and analyzed by flow cytometry. IL-12 production is analyzed by ELISA according to the manufacturer (Mabtech, Nacka, Sweden). Plates are coated with 2 μg/ml of anti-IL-12 capture antibody o.n, then after blocking, 100 μl of BMDC culture supernatants are added at dilutions 1:1-1:128 for 2 hr at RT. Plates are thereafter incubated with biotinylated anti-IL-12 antibody 1 hr at room temperature, and the response is visualized as above for ELISA.

Statistical Analyses

FMCA data is processed by GraphPad Prism (GraphPad Software, Inc. San Diego, Calif.) with non-linear regression to a standard sigmoidal dose-response model. Zero and 100% cell survival are set as the maximum effect and the baseline, and IC50 (inhibitory concentration 50%) is estimated. For drugs not resulting in 50% reduction of cell survival, the IC50 was set to>the highest concentration tested.

Results

After removal of unbound cisplatin by gel filtration, the amount of cisplatin associated with RHCC was measured by inductively-coupled plasma optical emission spectrometry. The molar ratio between cisplatin and the RHCC tetramer ranged from 0.9-1.0, indicating that on average one cavity in each coiled-coil tetramer was occupied by cisplatin. The RHCC/C complex was stable in solution up to 12 hr, shown by repeated measurements during dialysis against cisplatin-free buffer. Thus, the RHCC/C is sufficiently stable in solution to be applied in vivo. However, the molar ratio declined to 0.5 after 24 hr, probably due to slow diffusion from the coiled-coil cavities.

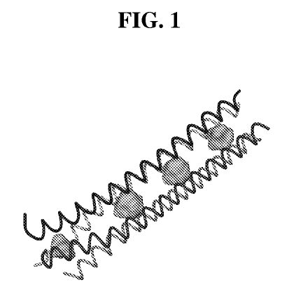

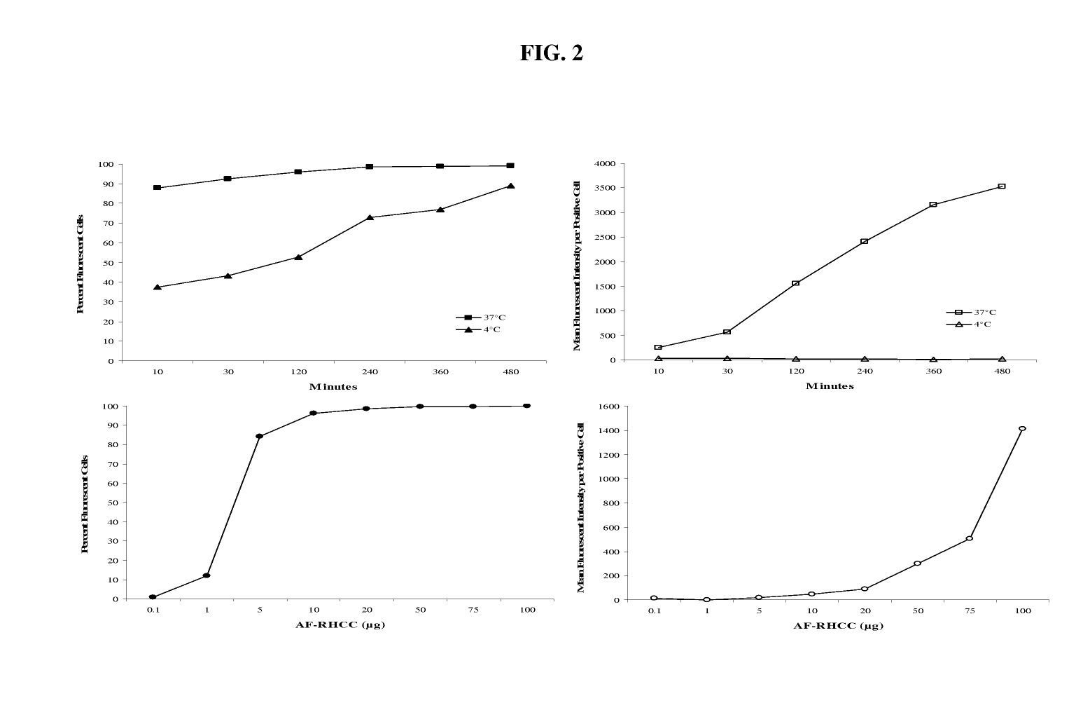

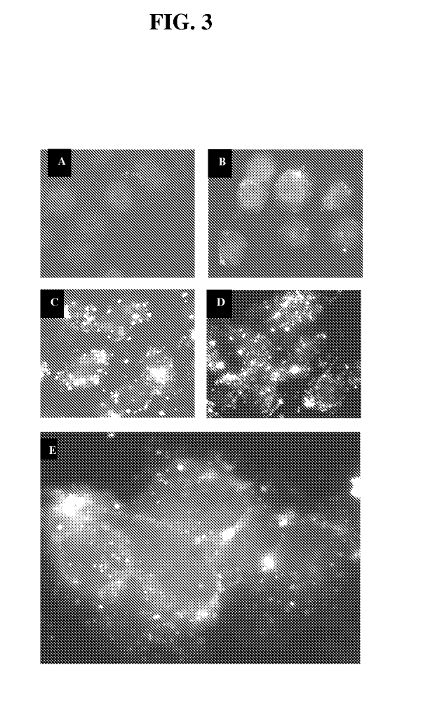

After incubation of AF-RHCC with FaDu-cells as described, the percentage of fluorescent cells, and the mean fluorescence of the cells, were estimated by flow cytometry. AF-RHCC bound better to FaDu-cells at 37° C. compared to at 4° C. After only 10 minutes at 37° C., 90% of the cells exhibited significant binding of AF-RHCC, and most cells (99%) still exhibited fluorescence 8 hours later (FIG. 2A). In contrast, at 4° C., binding was slower and with lower amount of AF-RHCC bound/cell (FIG. 1B). After incubation of 10 μg AF-RHCC with 5×104 FaDu-cells, most cells (96%) displayed binding of the protein, and with 50 μg, practically all cells (99.6%) bound AF-RHCC (FIG. 2C). However, the amount of AF-RHCC bound to each cell continued to rise and still no saturation was detected when incubating with 100 μg AF-RHCC (FIG. 2D). To elucidate if AF-RHCC stays bound on the cell surface or if it enters the cells, fluorescent- and confocal laser scanning microscopy was utilized after incubation of FaDu-cells with AF-RHCC for 10 min-8 hours, at 4° C. or 37° C. Incubation at 37° C. for 10-60 min resulted in a barely detectable fluorescent signal in the conventional fluorescent microscope (FIGS. 3A and B), while 4-8 hr of incubation gave a spotty pattern, suggesting AF-RHCC to be inside the cells (FIGS. 3C and D). Confocal laser scanning microscopy verified that the staining was in the cytoplasm of the cells, and that the staining was spotty, indicating uptake of the protein into intracellular vesicles (FIG. 3E). Almost no fluorescence was seen bound to or associated with cells incubated with AF-RHCC at 4° C. (data not shown).

Thus, RHCC was shown to bind to and enter the cytoplasm of FaDu-cells at 37° C., and the results from the flow cytometric binding assays showed that RHCC bound to almost 100% of the cells after a very short incubation time in vitro. The binding appeared unusually fast at first, indicating that the protein would possibly not have enough time to reach the tumor in vivo; however, when examining the amount of RHCC bound by the cells in this assay, it became apparent that a high proportion of free protein remained outside the cells after several hours of incubation. These data thus demonstrate that the protein binds to cells very efficiently, and that the uptake is not too fast for in vivo administration. The fact that the binding of RHCC to cells was so much more efficient at 37° C. compared to at 4° C., as seen in the flow cytometric experiments, may indicate an active energy-dependent mechanism for cellular uptake. As seen in the microscopic experiments, the uptake of RHCC into cells at 4° C. was almost non-existant, while at 37° C., much of the protein could be seen inside the cell cytoplasm in the confocal microscope. The spotty pattern may indicate an uptake in a specific subcellular compartment, e.g. lysosomes.

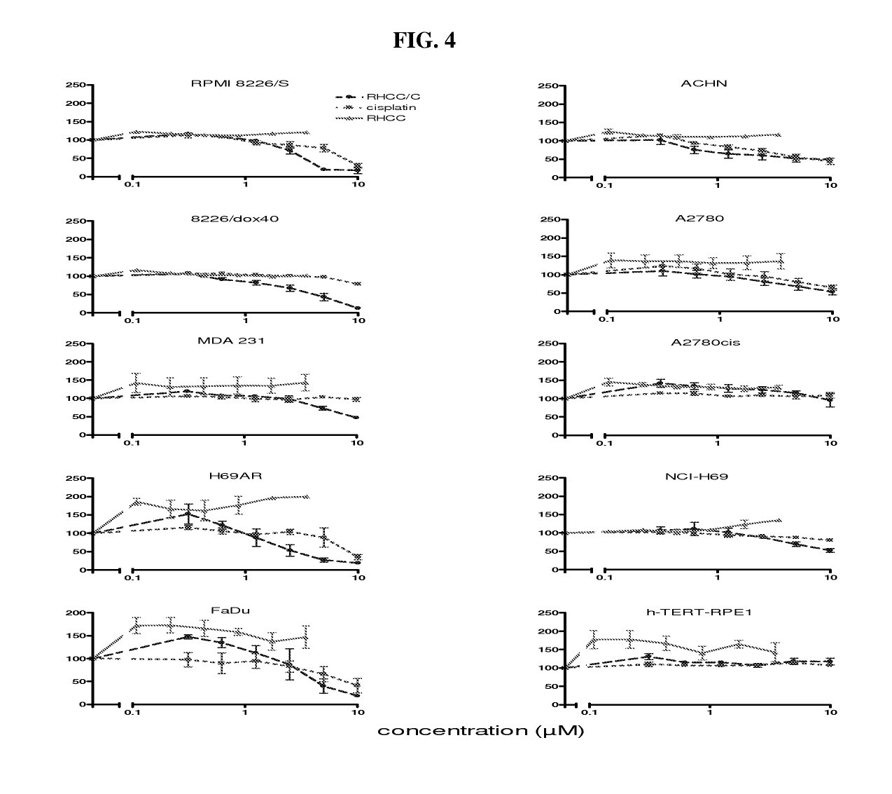

The cytotoxicity of RHCC, RHCC/C and cisplatin, cisplatin-sensitive and resistant cell lines were tested, as well as cells originating from tumor types currently treated with cisplatin. A dose-dependent decrease of viability of tumor cells from different cell lines was observed for RHCC/C and cisplatin in most of the evaluated cell lines. FIG. 4 displays the concentration-effect curves in all cell lines studied and Table I shows the corresponding IC50-values.

TABLE I
Estimated IC₅₀ (log IC₅₀ ± SEM) of
RHCC/C and cisplatin in 10 cell lines*

| | RHCC/C | Cisplatin |
|---|---|---|
| | IC₅₀ (μM) | IC₅₀ (μM) |
| Cell line | (95% confidence interval) | (95% confidence interval) |
| RPMI 8226/S | 3.37 (2.7-4.1) | 7.44 (6.0-9.1) |
| 8226/dox40 | 3.82 (3.1-4.7) | >10 |
| MDA 231 | 9.16 (7.3-11.5) | >10 |
| H69AR | 3.08 (1.8-5.4) | 8.50 (6.7-10.8) |
| FaDu | 4.66 (2.6-8.3) | 7.90 (4.2-14.8) |
| ACHN | 6.49 (2.8-15.3) | 6.90 (4.8-9.9) |
| A2780 | >10 | >10 |
| A2780cis | >10 | >10 |
| NCI-H69 | >10 | >10 |
| h-TERT-RPE1 | >10 | >10 |

*Where RHCC/C or cisplatin treatment did not result in 50% reduction of cell survival at the highest concentration tested (10 μM), the IC₅₀ was set to >10 μM.

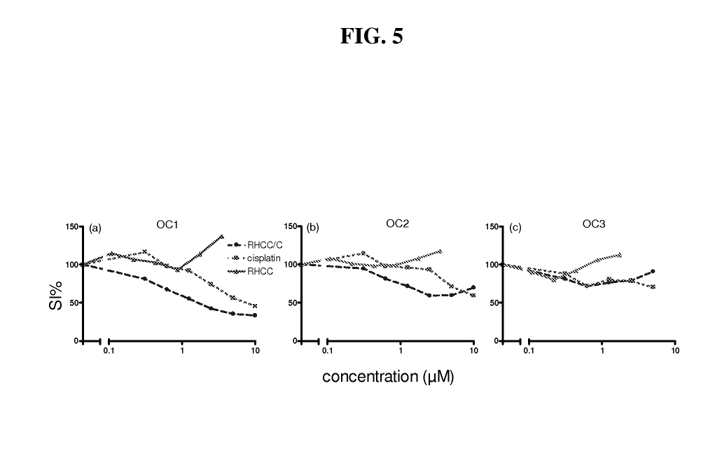

RHCC was nontoxic at the tested concentrations. Generally, RHCC/C and cisplatin had qualitatively similar cytotoxic effects in all the tested cell types. However, RHCC/C showed significantly higher effect than cisplatin in the myeloma cell line, RPMI 8226/S (P<0.0001), its sub-line 8226/dox40 (where IC50 for cisplatin could not be estimated), the adenocarcinoma breast cancer cell line MDA 231 (where IC50 for cisplatin could not be estimated), and the small-cell lung cancer sub-line H69AR (P<0.0015). RHCC/C and cisplatin displayed similar activity, with a tendency of RHCC/C to be slightly more efficient in the human head and neck squamous cell carcinoma cell line FaDu, the renal adenocarcinoma primary resistant cell line ACHN, the ovarian carcinoma cell lines A2780 and A2780cis, and the small-cell lung cancer cell line NCI H69. The normal epithelial telomerase immortalized hTERT-RPE1 cell line was not sensitive to RHCC/C or cisplatin. FIG. 5 illustrates the concentration-response curves in tumor cell samples from the three patients with ovarian carcinoma (OC1-OC3). RHCC/C showed higher activity in two patient tumor cell samples compared to cisplatin (FIGS. 5A and B), while both had a similar effect in tumor cells from the third patient (FIG. 5C).

The FMCA method has been extensively used for determining drug activity on human tumor cell lines as well as on primary tumor cells from patients with leukaemia and solid tumor malignancies. Moreover, results of different drugs tested with the FMCA on primary human tumor cultures have correlated very well with the clinical activity profile of that particular drug. In the current study, RHCC was not toxic at the tested concentration to any of the studied cell types. Moreover, RHCC/C and cisplatin, when used at equimolar cisplatin concentrations, induced similar cytotoxic effects against the different tumor types. In fact, for many cell lines, RHCC/C was even more active than cisplatin, as well as for primary human tumor cells from ovarian cancer patients. It is remarkable also that RHCC/C was more effective than cisplatin in two drug resistant cell lines; the myeloma 8226/dox40 and the small-cell lung cancer H69AR cell lines, supporting a different mode of entry or action. As mentioned above, RHCC alone had no cytotoxic effect in any of the cell lines and patient samples tested, or in the in vivo mouse tumor model, but its potentiating effect on cisplatin activity demonstrates a positive influence on the capability of an anti-tumor drug to penetrate the tumor cells. Furthermore, these in vitro results indicate that coupling of cisplatin to RHCC does not attenuate the cytotoxic effect of cisplatin.

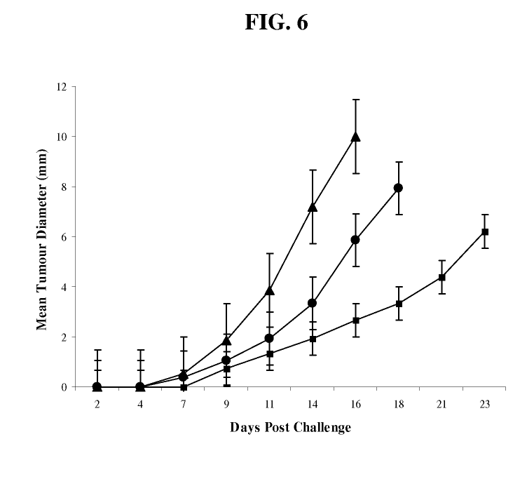

The SCID-mice challenged with FaDu-cells, and treated one week later with conventional cisplatin treatment, RHCC/C, or NaCl as a negative control shows the cytotoxic effect of cisplatin was retained and exhibited in vivo after coupling to RHCC (FIG. 6), and there was no significant alteration in weight observed in any animal during the experiment (data not shown). In a separate experiment, where mice were treated with RHCC alone, it could be seen that RHCC it self had no effect on tumor reduction (data not shown).

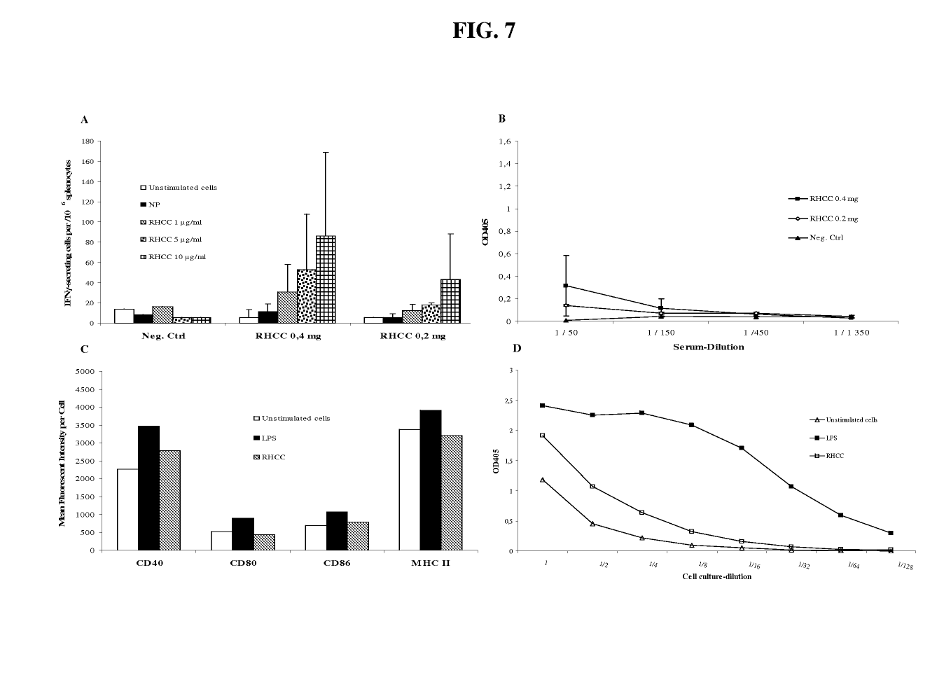

Injection of RHCC into Balb/c mice, co-cultured with murine DCs, showed RHCC induced a slight production of specific CD8+ T cells as seen in an IFNγ-EliSpot (FIG. 7A), but no significant antibody response in the ELISA (FIG. 7B). RHCC induced a very marginal maturation of DCs as seen from flow cytometry and ELISA experiments, with only a weak increase in CD40 expression and IL-12 production, and no increase in the other tested maturation markers CD80, CD86 and MHC Class II, as compared to that in unstimulated cells (FIGS. 7C and D). However, it is important to note that non-endotoxin purified RHCC induced a high unspecific reactivity in all immune response assays (data not shown). However, it should be mentioned that the RHCC preparation must be purified extensively from endotoxins in order to not obtain a broad unspecific immune response. Mice showed no signs of illness with regard to viability or alterations in weight after injection with endotoxin purified RHCC.

In summary, RHCC can bind to and enter into cells, and when purified from endotoxins, it does not induce a major immune response in vivo. Moreover, RHCC can deliver anti-tumor drugs into tumor cells and retain, or even also enhance, the cytotoxic potential of the drugs against a variety of tumor cell lines.

The methods of the present invention have been described with reference to specific embodiments and the Example demonstrates specific aspects of the invention. However, it will be appreciated that additional embodiments, aspects, variations and modifications of the invention can be effected by a person of ordinary skill in the art without departing from the scope of the invention as claimed.

What is claimed is:

1. A method of treating tumor cells in an individual, comprising administering to the individual a drug having anti-tumor effect, wherein the drug is delivered into the tumor cells via Right-Handed Coiled-Coil (RHCC) protein or a fragment or variant thereof.

2. The method of claim 1, wherein the drug is delivered into the tumor cells via RHCC protein.

3. The method of claim 1, wherein the drug comprises a metal-containing drug.

4. The method of claim 3, wherein the metal-containing drug comprises a platinum-containing drug.

5. The method of claim 4, wherein the metal-containing drug comprises at least one platinum-containing drug selected from the group consisting of Cisplatin, Carboplatin, Nedaplatin, Oxaliplatin, ZD0473, JM216, Lobaplatin, trans-[PtCl₂(pyridine)₂] and trans-[Pt(OH)₂Cl₂ (NH₃)(NH₂—C₂H₅].

6. The method of claim 4, wherein the metal-containing drug comprises at least one second drug in addition to the platinum-containing drug.

7. The method of claim 1, wherein the drug comprises a protein or peptide drug.

8. The method of claim 1, wherein the drug comprises a drug selected from the group consisting of actinomycins, anthracyclines, taxanes, antibiotics, tyrosine kinase inhibitors, cyclin dependent kinase inhibitors and histone deacetylase inhibitors.

9. The method of claim 1, wherein the metal-containing drug is delivered into the tumor cell in the absence of an added tag.

10. The method of claim 1, wherein the tumor cells are sarcoma, carcinoma, lymphoma or myeloma tumor cells.

11. The method of claim 1, wherein the tumor cells comprise ovarian carcinoma, testicular cancer, head tumor, neck tumor, breast cancer, colorectal cancer, urinary bladder cancer, renal cancer, small cell lung cancer, or non-small cell lung cancer cells.

12. The method of claim 1, wherein the drug is administered intravenously.

13. The method of claim 1, wherein the drug is administered by intraperitoneal delivery.

14. The method of claim 1, wherein the treatment provides a cytotoxic effect to the tumor cells.

15. The method of claim 1, wherein the treatment reduces a tumor size.

16. A method of treating tumor cells in an individual, comprising administering to the individual a platinum-containing drug having anti-tumor effect, wherein the drug is delivered into the tumor cells via Right-Handed Coiled-Coil (RHCC) protein or a fragment or variant thereof and in the absence of an added tag.

17. The method of claim 16, wherein the tumor cells comprise ovarian carcinoma, testicular cancer, head tumor, neck tumor, breast cancer, colorectal cancer, urinary bladder cancer, renal cancer, small cell lung cancer, or non-small cell lung cancer cells.

18. The method of claim 16, wherein the metal-containing drug comprises at least one platinum-containing drug selected from the group consisting of Cisplatin, Carboplatin, Nedaplatin, Oxaliplatin, ZD0473, JM216, Lobaplatin, trans-[PtCl₂(pyridine)₂] and trans-[Pt(OH)₂Cl₂ (NH₃)(NH₂—C₂H₅)].

19. The method of claim 18, wherein the metal-containing drug comprises Cisplatin.

20. The method of claim 1, wherein the drug is delivered into the tumor cells via RHCC protein.


SEQUENCE LISTING (2 sequences)

SEQ ID NO: 1 - protein, 52 aa, Artificial Sequence
  RHCC peptide including GS linker in N-terminal
GSIINETADDIVYRLTVIIDDRYESLKNLITLRADRLEMIINDNVSTILASI

SEQ ID NO: 2 - protein, 9 aa, Artificial Sequence
  LCVM derived peptide NP 118-126
RPQASGVYM